(19) United States
(12) Patent Application Publication
(10) Pub. No.: US 2006/0147553 A1
(43) Pub. Date: Jul. 6, 2006

(54) Method and apparatus for treatment of respiratory infections by nitric oxide inhalation

(76) Inventors: Christopher C. Miller, North Vancouver (CA); Richard Long, Edmonton (CA)

Correspondence Address: SIDLEY AUSTIN BROWN & WOOD LLP (LAIP GROUP), 555 W. FIFTH ST., SUITE 4000, LOS ANGELES, CA, 90013, US

(21) Appl. No.: 11/211,055

(22) Filed: Aug. 23, 2005

Related U.S. Application Data
(63) Continuation of application No. 09/762,152, filed on Feb. 1, 2001, now abandoned.

(30) Foreign Application Priority Data
Nov. 23, 1998 (CA) 2,254,645

(51) Int. Cl. A61K 33/00

(52) U.S. Cl. 424/718

(57) ABSTRACT

Methods for suppressing, killing, and inhibiting pathogenic cells, such as microorganisms associated with a respiratory infection within the respiratory tract of an animal are described. Methods include the step of exposing the pathogenic cells to an effective amount of nitric oxide, such as through inhalation of nitric oxide gas.

The application is a continuation application of and claims priority to U.S. application Ser. No. 09/762,152, filed on Feb. 1, 2001, which claims priority to International Patent Application No. PCT/CA99/01123, filed on Nov. 22, 1999, which claims priority to Canadian Application No. 2,254,645, filed on Nov. 23, 1998. Each of said applications are herein incorporated by reference in their entirety.

### FIELD OF THE INVENTION

The present invention relates to a method for suppressing pathogenic cells, as well as a method for the treatment of an animal, including a human, having pathogenic cells within its respiratory tract. These methods preferably comprise the exposure of the pathogenic cells to an effective amount of a source of nitric oxide, the nitric oxide source comprising nitric oxide or a compound or substance capable of producing nitric oxide and wherein the nitric oxide may have either an inhibitory or a cidal effect on such pathogenic cells.

Further, the present invention relates to the use of nitric oxide for suppressing pathogenic cells, the therapeutic use of nitric oxide for the treatment of an animal having pathogenic cells in its respiratory tract and a pharmaceutical composition for such treatment.

As well, in a preferred embodiment, the present invention relates to the use of nitric oxide in a gaseous form (NO) in the treatment of fungal, parasitic and bacterial infections, particularly pulmonary infection by mycobacterium tuberculosis. The invention also relates to an improved apparatus or device for the delivery, particularly pulsed-dose delivery, of an effective amount of nitric oxide for the treatment of microbial based diseases which are susceptible to nitric oxide gas. The device preferably provides nitric oxide replacement therapy at a desired dose for infected respiratory tract infections, or provides nitric oxide as a sterilizing agent for medical and other equipment, instruments and devices requiring sterilization.

### BACKGROUND OF THE INVENTION

In healthy humans, endogenously synthesized nitric oxide (NO) is thought to exert an important mycobacteriocidal or inhibitory action in addition to a vasodilatory action. There have been a number of ongoing, controlled studies to ascertain the benefits, safety and efficacy of inhaled nitric oxide as a pulmonary vasodilator. Inhaled nitric oxide has been successfully utilized in the treatment of various pulmonary diseases such as persistent pulmonary hypertension in newborns and adult respiratory distress syndrome. There has been no attempt, however, to reproduce the mycobacteriocidal or inhibitory action of NO with exogenous NO.

Further background information relating to the present invention may be found in the following references:
1. Lowenstein, C. J., J. L. Dinerman, and S. H. Snyder. 1994. Nitric oxide: a physiologic messenger” Ann. Intern. Med. 120:227-237.
2. The neonatal inhaled nitric oxide study group. 1997. Inhaled nitric oxide in full-term and nearly full-term infants with hypoxic respiratory failure. N. Engl. J. Med. 336:597-604.
3. Roberts, J. D. Jr., J. R Fineman, F. C. Morin III, et al. for the inhaled nitric oxide study group. 1997. Inhaled nitric oxide and persistent pulmonary hypertension of the newborn. N. Engl. J. Med. 336:605-610.
4. Rossaint, R., K. J. Falke, F. Lopez, K. Slama, U. Pison, and W. M. Zapol. 1993. Inhaled nitric oxide for the adult respiratory distress syndrome. N. Engl. J. Med. 328:399-405.
5. Rook, G. A. W. 1997. Intractable mycobacterial infections associated with genetic defects in the receptor for interferon gamma: what does this tell us about immunity to mycobacteria? Thorax. 52 (Suppl 3):S41-S46.
6. Denis, M. 1991. Interferon-gamma-treated murine macrophages inhibit growth of tubercle bacilli via the generation of reactive nitrogen intermediates. Cell. Immunol. 132:150-157.
7. Chan, J., R. Xing, R. S. Magliozzo, and B. R. Bloom. 1992. Killing of virulent Mycobacterium tuberculosis by reactive nitrogen intermediates produced by activated murine macrophages. J. Exp. Med. 175:1111-1122.
8. Chan, J., K. Tanaka, D. Carroll, J. Flynn, and B. R. Bloom. 1995. Effects of nitric oxide synthase inhibitors on murine infection with Mycobacterium tuberculosis. Infect. Immun. 63:736-740.
9. Nozaki, Y., Y. Hasegawa, S. Ichiyama, I. Nakashima, and K. Shimokata. 1997. Mechanism of nitric oxide—dependent killing of Mycobacterium bovis BCG in human alveolar macrophages. Infect. Immun. 65:3644-3647.
10. Canetti, G. 1965. Present aspects of bacterial resistance in tuberculosis. Am. Rev. Respir. Dis. 92:687-703.
11. Hendrickson, D. A., and M. M. Krenz. 1991. Regents and stains, P. 1289-1314. In Balows, A, W. J. Hausler Jr., K. L. Herrmann, H. D. Isenberg, and 1-1i. Shadomy (eds.), Manual of Clinical Microbiology, 5th ed., 1991. American Society for Microbiology, Washington, D.C.
12. Szabo, C. 1996. The pathophysiological role of peroxynitrite in shock, inflammation and ischemia—reperfusion injury. Shock. 6:79-88.
13. Stavert, D. M., and B. E. Lehnert. 1990. Nitrogen oxide and nitrogen dioxide as inducers of acute pulmonary injury when inhaled at relatively high concentrations for brief periods. Inhal. Toxicol. 2:53-67.
14. Hugod, C. 1979. Effect of exposure to 43 PPM nitric oxide and 3.6 PPM nitrogen dioxide on rabbit lung. mt. Arch. Occup. Environ. Health. 42:159-167
15. Frostell, C., M. D. Fratacci, J. C. Wain, R. Jones and W. M. Zapol. 1991. Inhaled nitric oxide, a selective pulmonary vasodilator reversing hypoxic pulmonary vasoconstriction. Circulation. 83:2038-2047.
16. BuIt, H., G. R. Y. Dc Meyer, F. H. Jordaens, and A. G. Herman. 1991. Chronic exposure to exogenous nitric oxide may suppress its endogenous release and efficacy. J. Cardiovasc. Pharmacol. 17:S79-S82.
17. Buga, G. M., J. M. Griscavage, N. E. Rogers, and L. J. Ignarro. 1993. Negative feedback regulation of endothelial cell function by nitric oxide. Circ. Res. 73:808-812
18. Assreuy, J., F. Q. Cunha, F. Y. Liew, and S. Moncada. 1993. Feedback inhibition of nitric oxide synthase activity by nitric oxide. Br. J. Pharmacol. 108:833-837.
19. O'Brien, L., J. Carmichael, D. B. Lowrie and P. W. Andrew. 1994. Strains of Mycobacterium tuberculosis differ in susceptibility to reactive nitrogen intermediates in vitro. Infect. Immun. 62:5187-5190.
20. Long, R., B. Maycher, A. Dhar, J. Manfreda, E. Hershfield, and N. R. Anthonisen. 1998. Pulmonary tuberculosis treated with directly observed therapy: serial changes in lung structure and function. Chest. 113:933-943.
21. Bass, H., J. A. M. Henderson, T. Heckscher, A. Oriol, and N. R. Anthonisen. 1968. Regional structure and function in bronchiectasis. Am. Rev. Respir. Dis. 97:598-609.

### SUMMARY OF THE INVENTION

In a first aspect of the invention, the invention relates to a method for suppressing pathogenic cells, and a method for treating an animal having pathogenic cells in its respiratory tract, utilizing a source of nitric oxide. More particularly, in the first aspect of this invention, the invention relates to a method for suppressing pathogenic cells comprising the step of exposing the pathogenic cells to an effective amount of a nitric oxide source. Further, the invention relates to a method for treating an animal having pathogenic cells in the respiratory tract of the animal comprising the step of delivering by the inhalation route to the respiratory tract of the animal an effective amount of a nitric oxide source.

In a second aspect of the invention, the invention relates to a use and a therapeutic use of a source of nitric oxide for suppressing or treating pathogenic cells. More particularly, in the second aspect of the invention, the invention relates to the use of an effective amount of a nitric oxide source for suppressing pathogenic cells exposed thereto. Further, the invention relates to the therapeutic use of an effective amount of a nitric oxide source for the treatment by the inhalation route of an animal having pathogenic cells in the respiratory tract of the animal. Preferably, as discussed further below, the present invention relates to the novel use of inhaled nitric oxide gas as an agent for killing bacterial cells, parasites and fungi in the treatment of respiratory infections.

In a third aspect of the invention, the invention relates to a pharmaceutical composition for use in treating an animal having pathogenic cells in its respiratory tract, which composition comprises a nitric oxide source. More particularly, in the third aspect of the invention, the invention relates to a pharmaceutical composition for use in the treatment by the inhalation route of an animal having pathogenic cells in the respiratory tract of the animal, the pharmaceutical composition comprising an effective amount of a nitric oxide source.

Finally, in a fourth aspect of the invention, the invention relates to an apparatus or device for supplying, delivering or otherwise providing a nitric oxide source. Preferably, the apparatus or device provides the nitric oxide source for the particular applications, methods and uses described herein. However, the apparatus or device may also be used for any application, method or use requiring the supply, delivery or provision of a nitric oxide source.

In all aspects of the invention, the nitric oxide source is preferably nitric oxide per se, and more particularly, nitric oxide gas. However, alternately, the nitric oxide source may be any nitric oxide producing compound, composition or substance. In other words, the nitric oxide source may be any compound, composition or substance capable of producing or providing nitric oxide, and particularly, nitric oxide gas. For instance, the compound, composition or substance may undergo a thermal, chemical, ultrasonic, electrochemical or other reaction, or a combination of such reactions, to produce or provide nitric oxide to which the pathogenic cells are exposed. As well, the compound, composition or substance may be metabolized within the animal being treated to produce or provide nitric oxide within the respiratory tract of the animal.

Further, in all aspects of the invention, the invention is for use in suppressing or treating any pathogenic cells. For instance, the pathogenic cells may be tumor or cancer cells. However, the pathogenic cells are preferably pathogenic microorganisms, including but not limited to pathogenic bacteria, pathogenic parasites and pathogenic fungi. More preferably, the pathogenic microorganisms are pathogenic mycobacteria. In the preferred embodiment, the pathogenic mycobacteria is M. tuberculosis.

Referring to the use of the nitric oxide source and method for suppressing pathogenic cells using the nitric oxide source, as indicated, the nitric oxide source is preferably nitric oxide per se. However, the nitric oxide source may be a compound, composition or substance producing nitric oxide. In either event, the pathogenic cells are suppressed by the nitric oxide. Suppression of the pathogenic cells by nitric oxide may result in either or both of an inhibitory effect on the cells and a cidal effect on the cells. However, preferably, the nitric oxide has a cidal effect on the pathogenic cells exposed thereto. Thus, it has been found that these aspects of the invention have particular application for the sterilization of medical and other equipment, instruments and devices requiring sterilization.

As well, the pathogenic cells may be exposed to the nitric oxide and the exposing step of the method may be performed in any manner and by any mechanism, device or process for exposing the pathogenic cells to the nitric oxide source, and thus nitric oxide, either directly or indirectly. However, in the preferred embodiment, the pathogenic cells are directly exposed to the nitric oxide. As a result, where desired, the effect of the nitric oxide may be localized to those pathogenic cells which are directly exposed thereto.

Similarly, the therapeutic use, method for treating and pharmaceutical composition for treatment all deliver the nitric oxide source to the pathogenic cells in the respiratory tract of the animal. The therapeutic use, method and composition may be used or applied for the treatment of any animal, preferably a mammal, including a human. Further, as indicated, the nitric oxide source in these instances is also preferably nitric oxide per se, however, the nitric oxide source may be a compound, composition or substance producing nitric oxide within the respiratory tract. In either event, the nitric oxide similarly suppresses the pathogenic cells in the respiratory tract of the animal. This suppression of the pathogenic cells may result in either or both of an inhibitory effect on the cells and a cidal effect on the cells. However, preferably, the nitric oxide has a cidal effect on the pathogenic cells in the respiratory tract exposed thereto.

As well, the pathogenic cells in the respiratory tract of the animal may be treated by nitric oxide and the delivering step of the therapeutic method may be performed in any manner and by any mechanism, device or process for delivering the nitric oxide source, and thus nitric oxide, either directly or indirectly to the respiratory tract of the animal. In the preferred embodiments of these aspects of the invention, the nitric oxide source is delivered directly by the inhalation route to the respiratory tract of the animal, preferably by either the spontaneous breathing of the animal or by ventilated or assisted breathing.

Further, in the preferred embodiments of these aspects of the invention, the pathogenic cells in the respiratory tract of the animal are treated by, and the delivering step of the therapeutic method is comprised of, exposing the pathogenic cells to the nitric oxide source, and thus nitric oxide, either directly or indirectly. More preferably, the pathogenic cells are directly exposed to the nitric oxide. As a result, where desired, the effect of the nitric oxide may be localized to those pathogenic cells which are directly exposed thereto within the respiratory tract of the animal.

In addition, in all aspects of the invention, an effective amount of the nitric oxide source is defined by the amount of the nitric oxide source required to produce the desired effect of the nitric oxide, either inhibitory or cidal, on the pathogenic cells. Thus, the effective amount of the nitric source will be dependent upon a number of factors including whether the nitric oxide source is nitric oxide per se or a nitric oxide producing compound, the desired effect of the nitric oxide on the pathogenic cells and the manner in which the pathogenic cells are exposed to or contacted with the nitric oxide. In the preferred embodiments of the various aspects of the invention, the effective amount of the nitric oxide source is the amount of nitric oxide required to have a cidal effect on the pathogenic cells exposed directly thereto. Thus, the effective amount for any particular pathogenic cells will depend upon the nature of the pathogenic cells and can be determined by standard clinical techniques. Further, the effective amount will also be dependent upon the concentration of the nitric oxide to which the pathogenic cells are exposed and the time period or duration of the exposure.

Preferably, the pathogenic cells are exposed to a gas or a gas is delivered to the respiratory tract of the animal being treated, wherein the gas is comprised of the nitric oxide source. More preferably, the pathogenic cells are exposed to a gas comprised of nitric oxide. For instance, the gas may be comprised of oxygen and nitric oxide for delivery by the inhalation route to the respiratory tract of the animal being treated.

Although in the preferred embodiments of the various aspects of the invention, any effective amount of nitric oxide may be used, the concentration of the nitric oxide in the gas is preferably at least about 25 parts per million. Further, the concentration of the nitric oxide in the gas is preferably less than about 100 parts per million. Most preferably, the concentration of the nitric oxide in the gas is between about 25 and 90 parts per million.

Although the pathogenic cells may be exposed to the gas for any time period or duration necessary to achieve the desired effect, the pathogenic cells are preferably exposed to the gas, or the gas is delivered to the respiratory tract of the animal, for a time period of at least about 3 hours. In the preferred embodiments of the various aspects of the invention, the pathogenic cells are exposed to the gas, or the gas is delivered to the respiratory tract of the animal, for a time period of between about 3 and 48 hours.

Finally, in the fourth embodiment of the invention, the apparatus or device is preferably comprised of a portable battery-operated, self-contained medical device that generates its own nitric oxide source, preferably nitric oxide gas, as a primary supply of nitric oxide. Further, the device may also include a conventional compressed gas supply of the nitric oxide source, preferably nitric oxide gas, as a secondary back-up system or secondary supply of nitric oxide.

Further, the device preferably operates to deliver nitric oxide in the gaseous phase to spontaneously breathing or to ventilated individual patients having microbial infections, by way of a specially designed nasal-cannula or a mask having a modified Fruman valve. In the preferred embodiment, nitric oxide gas is produced in cartridges through thermal-chemical, ultrasonic and/or electrochemical reaction and is released upon user inspiratory demand in pulsed-dose or continuous flow.

### BRIEF DESCRIPTION OF THE DRAWINGS

The nature and scope of the invention will be elaborated in the detailed description which follows, in connection with the enclosed drawing figures, in which:

FIG. 1 illustrates an airtight chamber for exposure of mycobacteria to varying concentrations of nitric oxide (NO) in tests of in vitro measurements of the cidal effects of exogenous NO;

FIG. 2 is a graphical representation of experimental data showing the relationship of percent kill of microbes to exposure time for fixed doses of NO;

FIG. 3a shows the external features of a pulse-dose delivery device for nitric oxide according to the present invention;

FIG. 3b illustrates schematically the internal working components of the device of FIG. 3a;

FIG. 4 is a schematic illustration of the specialized valve used to control the delivery of nitric oxide in a preset dosage through the disposable nasal cannula of a device according to the present invention; and

FIG. 5 is a schematic drawing of the mask-valve arrangement of a pulsed-dose nitric oxide delivery device according to the present invention.

### DETAILED DESCRIPTION OF THE INVENTION

Studies of the Applicant on the exposure of extra cellular M. tuberculosis to low concentrations of NO for short periods have led to the conclusion that exogenous NO exerts a powerful dose-dependent and time-dependent mycobacteriocidal action. Further, it may be inferred that the large population of extracellular bacilli in patients with cavitary pulmonary tuberculosis are also vulnerable to exogenous (inhaled) NO.

Measurements of Cidal Activity of Exogenous NO

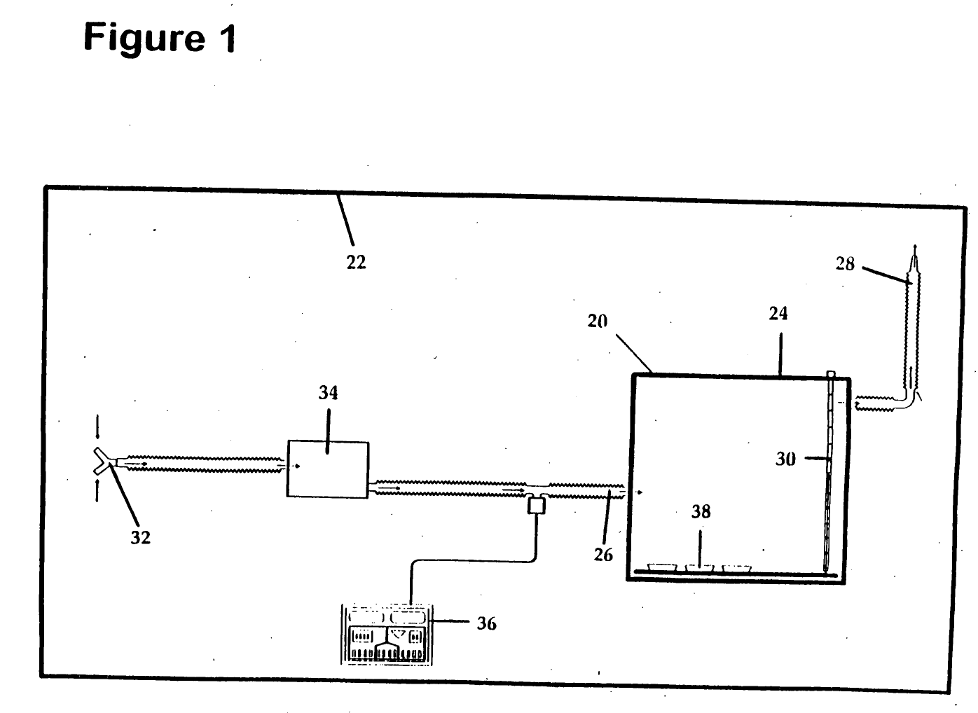

Referring to FIG. 1, to re-create a normal incubation environment that allowed for the exposure of mycobacteria to varying concentrations of NO, an airtight “exposure chamber” (20) was built that could be seated in a heated biological safetv cabinet (22). This chamber (20) measured 31×31×21 cm and is made of plexiglass. It has a lid (24) which can be firmly sealed, a single entry port (26) and a single exit port (28) through which continuous, low-flow, 5-10% CO₂ in air can pass, and a thermometer (30). A “Y” connector (32) in the inflow tubing allows delivery of NO, at predetermined concentrations, to the exposure chamber (20). Between the “Y” connector (32) and the exposure chamber (20) is a baffle box (34) which mixes the gases. Finally between the baffle box (34) and the exposure chamber (20) is placed an in-line NO analyzer (36), preferably a Pulmonox Sensor manufactured by Pulmonox Medical Corporation, Tofield, Alberta, Canada. This analyzer (36) continuously measures NO concentration in the gas mixture entering the exposure chamber (20).

The day before conducting the experiments, a precise quantity of actively growing virulent M. tuberculosis was plated on solid media (38) (Middlebrook 7H-10 with OADC enrichment) after careful dilution using McFarland nephelometry (1 in 10 dilution, diluted further to an estimated 103 bacteria/ml and using a 0.1 ml inoculate of this suspension) (see Reference No. 11 above under the Background of the Invention). Control and test plates were prepared for each experiment. Control plates were placed in a CO₂ incubator (Form a Scientific, Marietta, Ohio) and incubated in standard fashion at 37° C. in 5-10% CO₂ in air.

Test plates were placed in the exposure chamber (20) for a pre-determined period of time after which they were removed and placed in the incubator along with the control plates. The temperature of the exposure chamber (20) was maintained at 32-34° C. Colony counts were measured on control and test plates at 2, 3 and 6 weeks from the day of plating. Reported counts are those measured at three weeks expressed as a percentage of control.

Experiments were of two varieties: (1) those that involved exposure of the drug susceptible laboratory strain H37RV to fixed concentrations of NO, i.e. 0 (sham), 25, 50, 70 and 90 PPM for periods of 3, 6, 12, and 24 hours; and (2) those that involved exposure of a multidrug-resistant (isoniazid and rifampin) wild strain of M. tuberculosis to fixed concentrations of NO, i.e. 70 and 90 PPM for periods of 3, 6, 12 and 24 hours. One experiment at 90 PPM NO, that used both strains of M. tuberculosis, was extended to allow for a total exposure time of 48 hours. The NO analyzer (36) was calibrated at least every third experiment with oxygen (0 PPM of NO) and NO at 83 PPM.

Statistical Analysis

For each NO exposure time and NO concentration studied at least two, and in most cases three or four, separate experiments were performed with 3-6 exposure plates (38) per set. Colony counts performed on each exposure plate (38) were expressed as a percentage of the mean colony count of the matched non-exposed control plates. The values from all experiments at each NO concentration and exposure time were then averaged. These data were analyzed using two-way analysis of variance using the F statistic to test for independent effects of NO exposure time and NO concentration and of any interaction between them on the colony counts.

Experimental Results

A diagram of the incubation environment is shown in FIG. 1. This environment exactly simulated the usual incubation environment of M. tuberculosis in the laboratory, with the following exceptions: (1) the temperature of our exposure chamber (20) was maintained at 32-34° C. rather than the usual 37° C. to avoid desiccation of the nutrient media upon which the bacteria were plated; and (2) the test plates were openly exposed. That a stable and comparable incubation environment was reproduced was verified in four sham experiments using the H37RV laboratory strain of M. tuberculosis. Colony counts on plates (38) exposed to 5-10% CO₂ in air (0 PPM NO) at 32-34° C. in the exposure chamber (20) were not significantly different from those on control plates placed in the laboratory CO₂ incubator at 37° C., as shown in Table 1, below:

TABLE 1
COLONY COUNTS AFTER EXPOSURE OF THE LABORATORY
STRAIN (H37RV) OF M. TUBERCULOSIS TO VARYING
CONCENTRATIONS OF NITRIC OXIDE FOR PERIODS
OF 3, 6, 12 AND 24 HOURS
Colony Counts (Mean ± SE)
(expressed as percentage of control)

| | Exposure Time | | | |
|---|---|---|---|---|
| | (Hours) | | | |
| NO (PPM) | 3 | 6 | 12 | 24 |
| 0 | 107 ± 5(6)* | 100 ± 5(6) | 97 ± 9(6) | 105 ± 5(18) |
| 25 | 09 ± 6(12) | 109 ± 4(12) | 102 ± 3(12) | 66 ± 4(18) |
| 50 | 97 ± 5(12) | 96 ± 2(12) | 69 ± 3(12) | 41 ± 5(18) |
| 70 | 80 ± 10(7) | 63 ± 12(7) | 58 ± 12(11) | 21 ± 6(11) |
| 90 | 101 ± 15(11) | 67 ± 7(11) | 64 ± 7(14) | 15 ± 3(15) |

*Numbers in brackets refer to the number of plates prepared for each NO concentration at each time interval.

Seventeen experiments of the first variety, where plates (38) inoculated with a 0.1 ml suspension of 10³ bacteria/ml of the H37RV strain of M. tuberculosis were exposed to a fixed concentration (either 0, 25, 50, 70 or 90 PPM) of NO for increasing periods of time (3, 6, 12, and 24 hours) were performed. The results have been pooled and are outlined in Table 1. There were both dose and time dependent cidal effects of NO that were very significant by two-way ANOVA (F ratio 13.4, P<0.001; F ratio 98.1, P<0.0001 respectively) and there was also a statistically significant interactive effect on microbial killing efficacy (F ratio 2.03, P<0.048). Although there was some variability in the percentage killed from experiment to experiment, increasing the standard error of the pooled data, the dose and time effect were highly reproducible. Only one control and one test (12 hour) plate at 90 PPM were contaminated. That the effect of NO was cidal and not inhibitory was confirmed by the absence of new colony formation beyond three weeks.

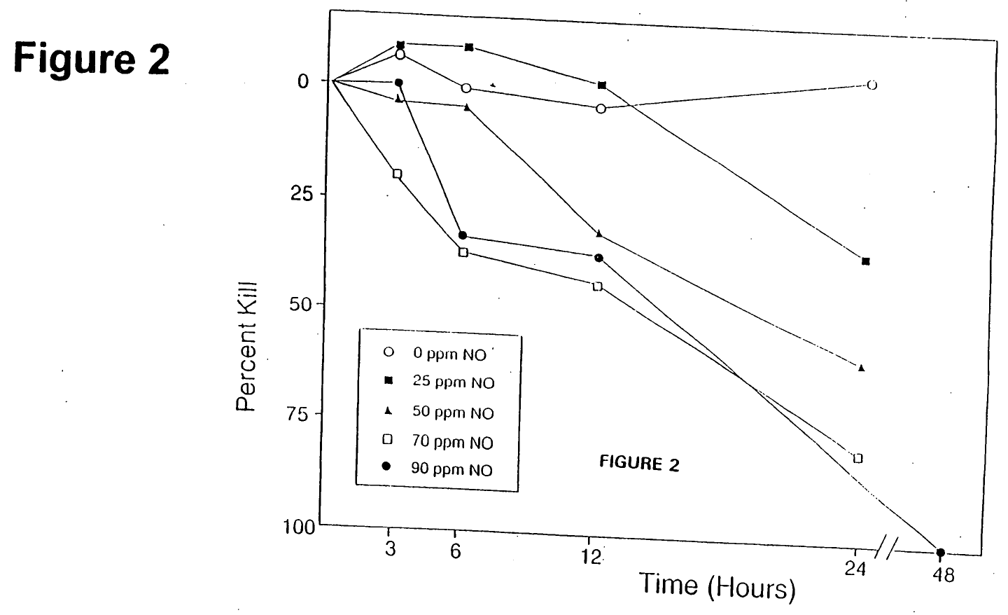

As described in FIG. 2, the response to a fixed dose of NO was relatively linear with the slope of the line relating exposure time to percent kill increasing proportionally with the dose. Dose-related microbial killing did not appear to increase above 70 PPM NO, since colony counts at 70 and 90 PPM were indistinguishable. At 24 hours of NO exposure at both the 70 and 90 PPM NO levels, more than one third of the exposed plates were sterile. One experiment at 90 PPM NO was extended to allow for a total exposure time of 48 hours; all of these plates were sterile (see FIG. 2 and Table 2 below)

TABLE 2
COLONY COUNTS AFTER EXPOSURE OF A
MULTIDRUG-RESISTANT WILD STRAIN OF M. TUBERCULOSIS
TO NITRIC OXIDE FOR PERIODS OF 3, 6, 12, 24 AND 48 HOURS
Colony Counts (Mean ± SE)
(expressed as percentage of control)

| | Exposure Time (Hours) | | | | |
|---|---|---|---|---|---|
| NO (PPM) | 3 | 6 | 12 | 24 | 48 |
| 70 | 113 ± 2(4) | 75 ± 4(4) | 85 ± 10(4) | 66 ± 4(4) | |
| | | | 50 ± 25(4) | 10 ± 5(4) | |
| 90 | 97 ± 11(2) | 91 ± 11(2) | 71 ± 8(2) | 36 ± 10(2) | |
| | | | 59 ± 4(4) | 32 ± 3(4) | 0 ± 0(4) |
| | | | 79 ± 5(4)^{#} | 20 ± 3(4)^{#} | 0 ± 0(4)^{#} |

*Each series represents an individual experiment; numbers in brackets refer to the number of plates prepared for each experiment at each time interval.
^{#}These results refer to the H37RV laboratory strain.

Four experiments of the second variety, where plates inoculated with a 0.1 ml suspension of 10³ bacteria/ml of a multidrug-resistant wild strain of M. tuberculosis, were exposed to a fixed concentration (either 70 or 90 PPM) of NO for increasing periods of time (3, 6, 12 and 24 hours) were performed, two at each of 70 and 90 PPM NO. Again there was a significant dose and time dependent cidal effect (see Table 2 above). Although the percent kill at 24 hours was less than that observed with the H37RV strain, when an inoculum of this strain was exposed to 90 PPM NO for a period of 48 hours there was also 100% kill.

### CONCLUSION

Using an in vitro model in which the nitric oxide concentration of the incubation environment was varied, we have demonstrated that exogenous NO delivered at concentrations of less than 100 PPM exerts a powerful dose and time dependent mycobacteriocidal action. When an inoculate of M. tuberculosis that yielded countable colonies (0.1 ml of a suspension of 10³ bacteria/ml) was plated on nutrient rich media and exposed to exogenous NO at 25, 50, 70 and 90 PPM for 24 hours there was approximately 30, 60, 80 and 85% kill, respectively. Similarly when plates of the same inocula were exposed to a fixed concentration of exogenous NO, for example 70 PPM, for increasing durations of time, the percentage of kill was directly proportional to exposure time; approximately 20, 35, 40 and 80% kill at 3, 6, 12 and 24 hours, respectively.

Of added interest, the dose and time dependent mycobacteriocidal effect of NO was similar for both the H37RV laboratory strain and a multidrug-resistant (isoniazid and rifampin) wild strain of M. tuberculosis, (after 24 and 48 hours exposure to 90 PPM NO, there was 85 and 100% kill and 66 and 100% kill of the two strains, respectively) expanding the potential therapeutic role of exogenous NO and suggesting that the mechanism of action of NO is independent of the pharmacologic action of these cidal drugs.

The dominant mechanism(s) whereby intracellular NO, known to be produced in response to stimulation of the calcium-independent inducible nitric oxide synthase, results in intracellular killing of mycobacteria is still unknown (see Reference No. 5 above under the Background of the Invention). Multiple molecular targets exist, including intracellular targets of peroxynitrite, the product of the reaction between NO and superoxide (see Reference No. 12 above under the Background of the Invention). Whatever the mechanism(s), there is evidence that NO may be active not just in murine but also in human alveolar macrophages (see References No. 6-9 above under the Background of the Invention), and furthermore that this activity may be critical to the mycobacteriocidal action of activated macrophages. Whether macrophase inducible NOS produces NO that has extracellular activity is not known but it is reasonable to expect that a measure of positive (mycobacteriocidal) and negative (tissue necrosis) activity might follow the death of the macrophase itself.

The relative ease with which NO may be delivered exogenously, and its theoretical ability to rapidly destroy the extracellular population of bacilli in the patient with sputum smear positive pulmonary tuberculosis, especially drug-resistant disease, have great clinical appeal.

Primary Unit of the NO Post-Delivery Device

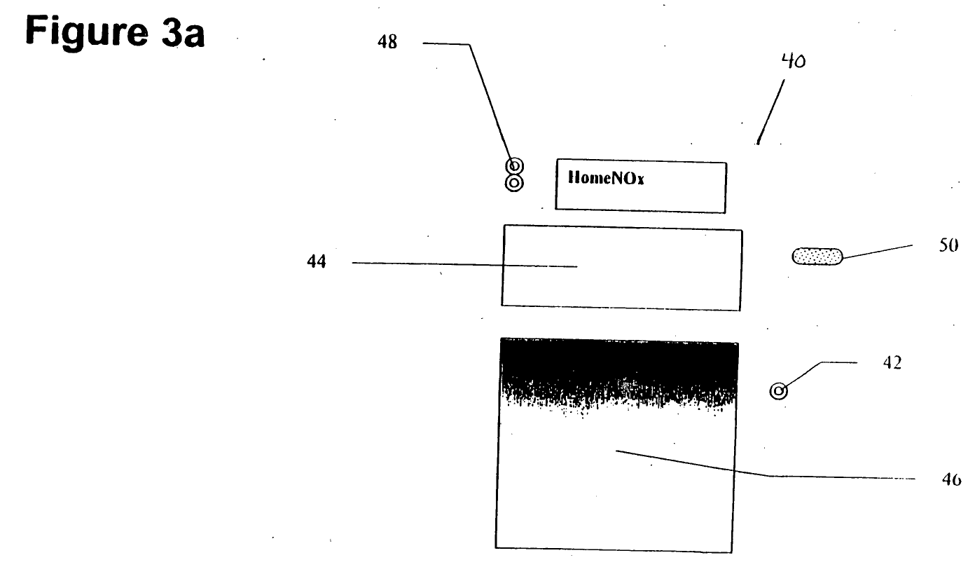

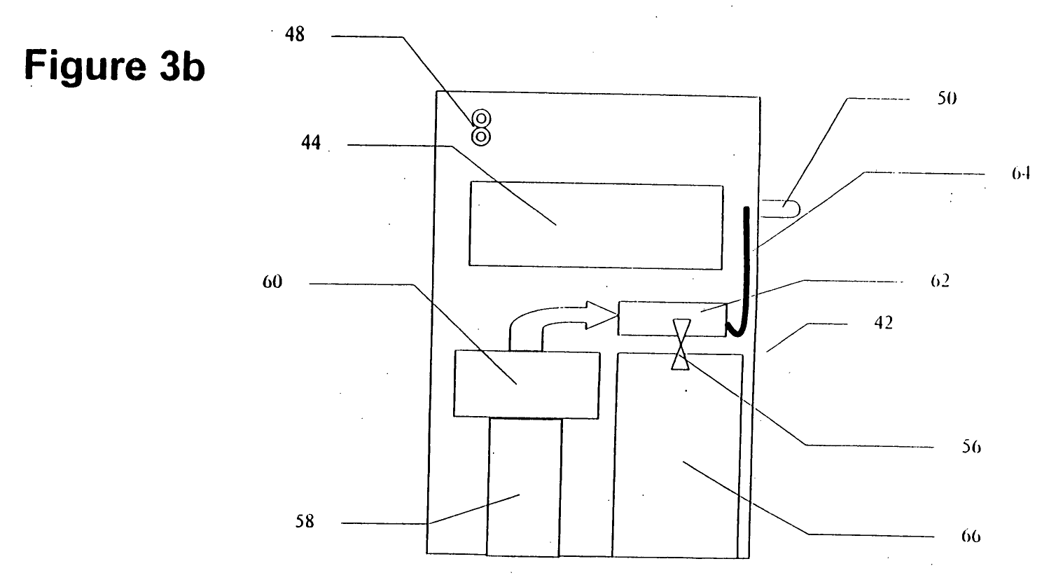

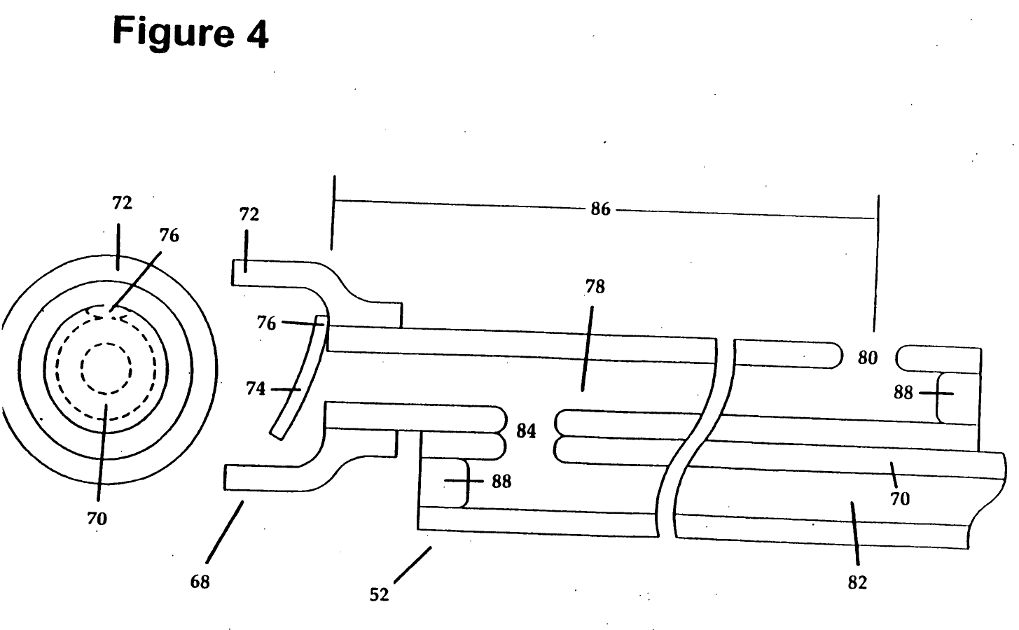

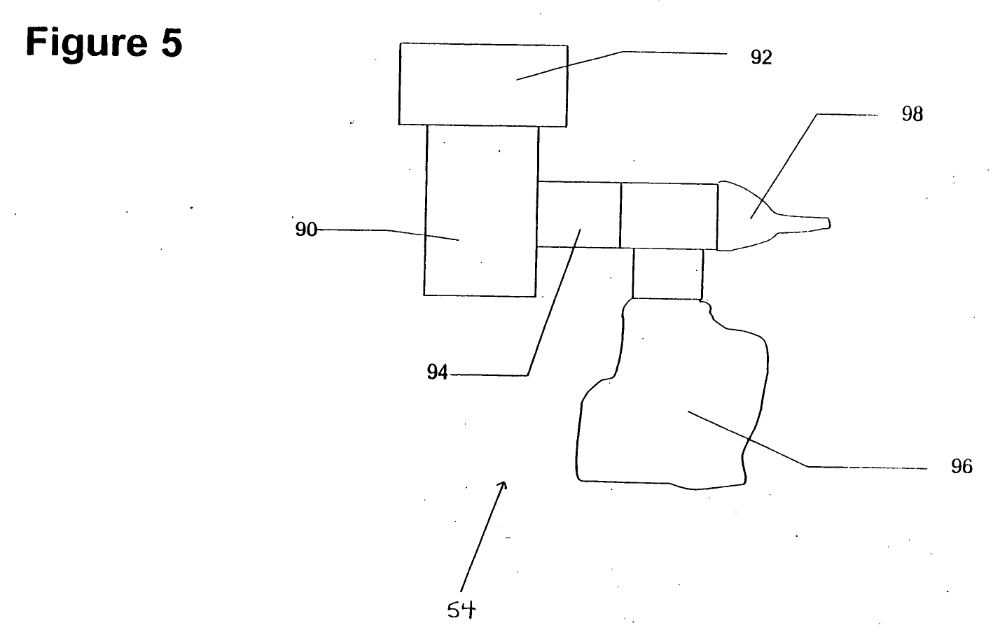

Referring to FIGS. 3a and 3b, the main unit (40) provides a small enclosure designed to hang on a belt. An A/C inlet (42) provides an electrical port to provide power to an internal rechargeable battery which powers the unit (40) if required. The user interface provides a multi-character display screen (44) for easy input and readability. A front overlay (46) with tactile electronic switches allow easy input from user to respond to software driven menu commands. LED and audible alarms (48) provide notification to user of battery life and usage. A Leur-type lock connector (50) or delivery outlet establishes communication with the delivery line to either the nasal cannula device (52) shown in FIG. 4 or the inlet conduit on the modified Fruman valve (54) shown in FIG. 5.

More particularly, referring to FIG. 3b, the main unit (40) houses several main components. A first component or subassembly is comprised of an electronic/control portion of the device. It includes a microprocessor driven proportional valve or valve system (56), an alarm system, an electronic surveillance system and data input/output display system and electronic/software watch dog unit (44).

A second component or subassembly includes one or more disposable nitric oxide substrate cartridges (58) and an interface mechanism. A substrate converter system or segment (60) processes the primary compounds and converts it into pure nitric oxide gas. The gas then flows into an accumulator stable (62) and is regulated by the proportional valve assembly (56) into a NO outlet nipple (64).

A third component or subassembly is comprised of a secondary or backup nitric oxide system (66). It consists of mini-cylinders of high nitric oxide concentration under low-pressure. This system (66) is activated if and when the primary nitric oxide source (58) is found faulty, depleted or not available.

Nasal Cannula Adjunct

Referring to FIG. 4, there is shown a detailed drawing of a preferred embodiment of a valve (68) used to control the delivery of nitric oxide in a preset dosage through a disposable nasal cannula device (52) as shown. The valve (68) is controlled by the natural action of spontaneous respiration by the patient and the dosage is preset by the physical configuration of the device (52).

The device (52) including the valve (68) is constructed of dual lumen tubing (70). The internal diameter of the tubing (70) depends on the required dosage. The tubing (70) is constructed of material compatible with dry nitric oxide gas for the duration of the prescribed therapy. This tubing (70) is glued into the nasal cannula port (72).

The valve (68) is preferably comprised of a flexible flapper (74) that is attached by any mechanism, preferably a spot of adhesive (76), so as to be positioned over the supply tube (70). The flapper (74) must be sufficiently flexible to permit the valve action to be effected by the natural respiration of the patient. When the patient breathes in, the lower pressure in the nasal cannula device (52) causes the flapper (74) of the valve (68) to open and the dry gas is delivered from a reservoir (78) past the flapper (74) and into the patient's respiratory tract. When the patient exhales, positive pressure in the nasal cannula device (52) forces the flapper (74) of the valve (68) closed preventing any delivered gas entering the respiratory tract.

The supplied gas is delivered at a constant rate through the supply tube (70). The rate must be above that required to deliver the necessary concentration to the patient by filling the supply reservoir (78) up to an exhaust port (80) in the supply tube (70) during expiration. When the patient is exhaling the flapper (74) is closed and the supply gas feeds from a supply line (82) through a cross port (84) into the reservoir or storage chamber (78). The length of the reservoir chamber (78) given as dimension (86) determines the volume of gas delivered when the patient inhales. Inhaling opens the flapper (74) of the valve (68) and causes the reservoir chamber (78) to be emptied.

During exhalation when the flapper (74) is closed and the reservoir chamber (78) is filling, any excess gas exhausts through the exhaust port (80). During inhalation when the reservoir chamber (78) is emptied, the reservoir chamber (78) is displaced with atmospheric air through the exhaust port (80). There will continue to be supply gas from the supply line (82) through the cross port (84) during inhalation and this amount must be figured into the total delivered gas to determine the actual dosage. The tubing lumens (70) include various plugs (88) to direct the flow.

Mask/Valve Adjunct

Referring to FIG. 5, there is shown a further embodiment of a nitric oxide valve (54) which is a modification and improvement of a Non-rebreathing valve for gas administration, referred to as a “Modified Fruman Valve,” as shown and particularly described in U.S. Pat. No. 3,036,584 issued May 29, 1962 to Lee.

More particularly, the within invention specifically redesigns the Modified Fruman Valve for use in inhaled nitric oxide therapy. Specifically, in the preferred embodiment shown in FIG. 5, one end of a valve body (90) or valve body chamber is comprised of or includes a mask or mouth-piece (not shown) attached thereto. The connection is preferably standardized to a 22 mm O.D. to facilitate the attachment of the mask or mouth-piece. The other end of the valve body (90) is comprised of or provides an exhaust port (92). The exhaust port (92) entrains ambient air during the latter portion of inspiration and dilutes the nitric oxide coming from an inlet conduit (94).

The resultant nitric oxide concentration in the valve body (90) is determined by the dilutional factors regulated by the valve (54), tidal volume and the nitric oxide concentration in an attached flexed bag (96), being a fixed reservoir bag. The inlet conduit (94) is preferably spliced for the attachment of the small flexed bag (96). The purpose of the bag (96) is to act as a reservoir for nitric oxide gas. Further, an opening of the inlet conduit (94) is preferably modified to facilitate the attachment or connection of the inlet conduit (94) to a supply hose emanating from a nitric oxide supply chamber. Specifically, the opening of the inlet conduit (94) is preferably comprised of a knurled hose barb connector (98).

While the invention herein disclosed has been described by means of specific embodiments and applications thereof, numerous modifications and variations could be made thereto by those skilled in the art without departing from the scope of the invention set forth in the claims.

What is claimed is:

1. A method of killing or inhibiting the proliferation of extracellular microorganisms associated with a respiratory infection within the respiratory tract of an animal, the method comprising the steps of:
providing a flow-controlled source of nitric oxide gas;
delivering the nitric oxide gas to the animal's respiratory tract through inhalation; and
wherein the inhalation of nitric oxide gas results in direct exposure of the inhaled nitric oxide gas to the microorganisms within the respiratory tract.

2. The method of claim 1, wherein the nitric oxide gas is delivered through spontaneous breathing of the animal.

3. The method of claim 1, wherein the nitric oxide gas is delivered through a ventilator.

4. The method of claim 1, wherein the nitric oxide gas flows from a pressurized cylinder containing nitric oxide gas.

5. The method of claim 1, further comprising the step of diluting the nitric oxide gas, prior to inhalation.

6. The method of claim 1, wherein the flow of nitric oxide gas from the flow-controlled source of nitric oxide gas is regulated by a valve.

7. The method of claim 6, wherein the nitric oxide gas flows from a nitric oxide substrate source containing a compound capable of producing nitric oxide gas.

8. The method of claim 1, wherein the nitric oxide gas is delivered in a continuous flow.

9. The method of claim 1, wherein the nitric oxide gas is delivered in pulsed-doses.

10. A method of suppressing a respiratory infection associated with microorganisms within the respiratory tract of an animal, the method comprising the steps of:
providing a pressurized source of nitric oxide gas;
diluting the nitric oxide gas;
delivering the diluted nitric oxide gas to the animal by inhalation resulting in direct exposure of the inhaled nitric oxide gas to microorganisms within the animal's respiratory tract.

11. The method of claim 10, wherein the nitric oxide gas is diluted with air.

12. The method of claim 10, further comprising the steps of monitoring a concentration of the nitric oxide gas.

13. The method of claim 10, wherein the nitric oxide gas is delivered through a ventilator.

14. The method of claim 10, further comprising the step of providing for a nasal cannula to interface with the animal wherein nitric oxide gas is inhaled through the nasal cannula.

15. The method of claim 10, wherein the nitric oxide gas is delivered in pulsed-doses.

16. The method of claim 10, wherein the nitric oxide gas is diluted with an oxygen containing gas.

17. A method for treating an animal having pathogenic microorganisms in the respiratory tract of the animal comprising the step of:
delivering nitric oxide gas to the animal by inhalation resulting in direct exposure of the inhaled nitric oxide gas to pathogenic microorganisms within the animal's respiratory tract,
wherein the nitric oxide gas is delivered in an amount effective to kill or inhibit the proliferation of said pathogenic microorganisms.

18. The method of claim 17, wherein the microorganisms are selected from the group consisting of pathogenic bacteria, pathogenic parasites and pathogenic fungi.

19. The method of claim 18, wherein the microorganisms are pathogenic mycobacteria.

20. The method of claim 17, wherein the animal is a human.

21. The method of claim 17, wherein the delivering step comprises directly exposing the pathogenic cells in the respiratory tract of the animal to said nitric oxide gas.

22. The method of claim 21, wherein said nitric oxide gas has a cidal effect on the pathogenic cells.

23. The method of claim 22, wherein the animal is a human.

24. The method of claim 17, wherein the delivering step comprises delivering a gas mixture comprising nitric oxide gas in a concentration of at least about 25 parts per million.

25. The method of claim 17, wherein the delivering step is comprises delivering a gas mixture comprising nitric oxide gas in a concentration of less than about 100 parts per million.

26. The method of claim 25, wherein the concentration of said nitric oxide in the gas mixture is between about 25 and 90 parts per million.

27. The method of any of claims 26, wherein the animal is a human.

28. The method of claim 27, wherein the gas mixture is delivered for a time period of at least about 3 hours.

29. The method of claim 27, wherein the gas mixture is delivered for a time period of between about 3 and 48 hours.
